SEQUENCE LISTING (2 sequences)

SEQ ID NO: 1 - protein, 101 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 2 - protein, 110 aa, Homo sapiens
MITATLNGEPAECLATVPGAAPAPPTWLEQLLSGGGVIYAEAEEDGDLQCLCVKTTSQVRPRHITSLEVIKAGPHCPTAQLIATLKNGRKICLDLQAPLYKKIIKKLLES

(19) United States
(12) Patent Application Publication
(10) Pub. No.: US 2025/0130243 A1
(43) Pub. Date: Apr. 24, 2025

(54) METHODS AND MATERIALS FOR IDENTIFYING AND TREATING VACCINE-INDUCED IMMUNE THROMBOTIC THROMBOCYTOPENIA

(71) Applicant: Mayo Foundation for Medical Education and Research, Rochester, MN (US)

(72) Inventor: Anand Padmanabhan, Rochester, MN (US)

(21) Appl. No.: 18/834,684

(22) PCT Filed: Jan. 26, 2023
(86) PCT No.: PCT/US2023/061341

§ 371 Date: Jul. 31, 2024

Related U.S. Application Data
(60) Provisional application No. 63/305,423, filed on Feb. 1, 2022.

(51) Int. Cl. G01N 33/68

(52) U.S. Cl. CPC G01N 33/6893; G01N 2333/521; G01N 2800/226; G01N 2800/52

(57) ABSTRACT

Methods and materials for identifying and/or treating mammals (e.g., humans) as having vaccine-induced immune thrombotic thrombocytopenia (VITT) are provided herein. In some cases, the methods and materials provided herein can be used to differentiate between VITT and spontaneous heparin-induced thrombocytopenia (s-HIT). Specifically, the method comprising contacting the sample from a mammal that (i) has thrombocytopenia, and/or thrombosis, and (ii) does not have heparin-induced thrombocytopenia (HIT) with an uncomplexed platelet factor 4 (PF4) polypeptide for detection.

### CROSS-REFERENCE TO RELATED APPLICATIONS

This application claims priority from U.S. Provisional Application Ser. No. 63/305,423, filed Feb. 1, 2022. The disclosure of the prior application is considered part of (and is incorporated by reference in) the disclosure of this application.

### STATEMENT OF FEDERALLY SPONSORED RESEARCH

This invention was made with government support under HL158932 awarded by the National Institutes of Health. The government has certain rights in the invention.

### SEQUENCE LISTING

This application contains a Sequence Listing that has been submitted electronically as an XML file named “07039-2119WO1.XML.” The XML file, created on Jan. 20, 2023, is 3013 bytes in size. The material in the XML file is hereby incorporated by reference in its entirety.

### TECHNICAL FIELD

This document relates to methods and materials for identifying and treating vaccine-induced immune thrombotic thrombocytopenia (VITT). For example, this document provides methods and materials that can be used to differentiate between VITT and spontaneous thrombotic thrombocytopenia (spontaneous HIT or s-HIT).

### BACKGROUND

Some COVID-19 vaccines have been associated with a severe thrombotic and thrombocytopenia adverse reaction characterized by platelet-activating antibodies that can bind to platelet-factor-4 (PF4), a chemokine that can form complexes with polyanions such as heparin and/or polyvinyl sulfonate. The reaction to the vaccines, referred to as vaccine-induced immune thrombotic thrombocytopenia (VITT) or thrombosis and thrombocytopenia syndrome (TTS), was first identified in 2021, and to date, has been noted with three COVID-19 vaccines: ChAdOx01 nCOV-19 (AstraZeneca, AZ), Ad26.COV2.S (Janssen Johnson & Johnson, J&J), and mRNA-1273 (Moderna). The risk of these reactions vary, with about 10 reactions per million vaccinated with AZ, about 3 to 4 per million vaccinated with J&J, and ultra-rare with mRNA vaccines, at a rate of about 1 reaction per about 165 million vaccinations. Preliminary studies suggested that VITT also may be induced by other vaccines (e.g., the Gardasil human papilloma virus vaccine; Johansen et al., J Thromb Haemost. 20(3):700-704, 2021).

VITT after COVID-19 vaccination is caused by antibodies against PF4, a member of the CXC chemokine family that has high affinity for heparin. The anti-PF4 antibodies that cause VITT are not well characterized, but they can be detected with at least two types of laboratory assays. The first type includes “functional” platelet-activation based assays such as the PF4-dependent P-selectin expression assay (PEA), the modified heparin induced platelet activation (HIPA) assay, and the PF4-induced platelet activation (PIPA) assay (see, e.g., Greinacher et al., N Engl J Med. 2021, 384(22):2092-2101; and George et al., Am J Hematol. E301-302). However, these assays are non-specific, detecting not only VITT antibodies, but also antibodies from heparin-induced thrombocytopenia (HIT) and spontaneous HIT (s-HIT) patients. s-HIT is a dangerous syndrome in which patients develop thrombosis and thrombocytopenia without proximate exposure to heparin. The second type of assay that can be used to detect pathogenic VITT antibodies is an antigen assay. For example, ELISA assays with PF4-polyanion targets that have been optimized to detect antibodies in HIT can also detect VITT with high sensitivity (see, e.g., Greinacher et al., supra; and George et al., supra). Like the functional testing, however, these antigen-based assays are non-specific, detecting antibodies associated with VITT, HIT, and s-HIT, as well as non-activating non-pathogenic PF4-polyanion ELISA-positive antibodies that are present in more than 4% of the general population with no obvious pathology (Hursting et al., Am J Clin Path. 2010, 134(5):774-780), and non-activating, non-pathogenic anti-PF4 antibodies that are commonly found in heparin-exposed individuals (Pouplard et al., Circulation. 1999, 99(19):2530-2536).

### SUMMARY

This document provides methods and materials for identifying and treating mammals (e.g., humans) having VITT, such that mammals with VITT can be distinguished from mammals with s-HIT, as well as from mammals that are PF4/polyanion ELISA positive and may have developed coincidental non-VITT related thrombocytopenia/thrombosis in the setting of vaccination, and from mammals that may have developed HIT after exposure to heparin in the setting of vaccination. As demonstrated herein, for example, an uncomplexed PF4 enzyme-linked immunosorbent assay (ELISA) can be used to specifically differentiate Ad26.COV2.S-associated VITT, ChAdOx1 nCOV-19-associated VITT, mRNA-1273-associated VITT, and human papillomavirus (HVP) 9-valent vaccine, recombinant (e.g., GARDASILR) associated VITT from HIT, s-HIT, and non-activating PF4/polyanion ELISA-positive but non-activating anti-PF4 antibodies that may be present in healthy individuals and in patients treated with heparin but without HIT. In addition to their specificity for identifying VITT, the methods and materials provided herein also have a high degree of sensitivity. PF4-polyanion ELISA testing also revealed that antibodies persisted for more than 5 months in Ad26.COV2.S-associated VITT patients, mediating mild persistent thrombocytopenia in some cases, despite becoming undetectable in functional testing during the same time frame.

Differentiating VITT from s-HIT has been particularly challenging from clinical and laboratory standpoints. In fact, evidence has suggested that VITT is a hybrid between two types of s-HIT, specifically post-orthopedic s-HIT and “medical” s-HIT (Warkentin and Greinacher, Thromb Res. 2021, 204:40-51). The methods described herein provide a solution to this diagnostic problem.

The ability to distinguish VITT from s-HIT also has patient management implications. For example, having the ability to identify mammals (e.g., humans) specifically as having VITT can allow those mammals to be properly identified and treated in an effective and reliable manner. As described herein, obtaining the correct diagnosis can send VITT patients down a different treatment and management path than the treatment and management path for individuals with s-HIT, HIT, or PF4/polyanion ELISA positive but non-activating anti-PF4 antibodies.

In general, one aspect of this document features a method for distinguishing vaccine-induced immune thrombotic thrombocytopenia (VITT) from spontaneous heparin-induced thrombocytopenia (s-HIT). The method can include, or consist essentially of, (a) contacting an uncomplexed PF4 polypeptide with a sample from a mammal that (i) has thrombocytopenia, thrombosis, or both thrombocytopenia and thrombosis, and (ii) does not have heparin-induced thrombocytopenia (HIT), wherein antibody-uncomplexed PF4 polypeptide complexes form if the sample contains VITT antibodies, and wherein antibody-uncomplexed PF4 polypeptide complexes do not form if the sample does not contain VITT antibodies, and (b) detecting the presence or absence of the antibody-uncomplexed PF4 polypeptide complexes, wherein the presence of the antibody-uncomplexed PF4 polypeptide complexes indicates that the mammal has VITT, and wherein the absence of the antibody-uncomplexed PF4 polypeptide complexes indicates that the mammal has s-HIT. The sample can be a blood sample. The mammal can be a human. The detecting can include using an enzyme-linked immunosorbent assay (ELISA). The method can further include, when the presence of the antibody-uncomplexed PF4 polypeptide complexes is detected, administering to the mammal intravenous immunoglobulin G (IVIg), a non-heparin anticoagulant, a corticosteroid, therapeutic plasma exchange (TPE) treatment, or a combination thereof to treat the VITT. The method can further include, when the presence of the antibody-uncomplexed PF4 polypeptide complexes is detected, monitoring the mammal for platelet counts or D-dimer levels to assess the severity or progression of the VITT.

In another aspect, this document features a method that includes, or consists essentially of, (a) contacting an uncomplexed PF4 polypeptide with a sample from a mammal that (i) has thrombocytopenia, thrombosis, or both thrombocytopenia and thrombosis, and (ii) does not have HIT, wherein antibody-uncomplexed PF4 polypeptide complexes form if the sample contains VITT antibodies, and wherein antibody-uncomplexed PF4 polypeptide complexes do not form if the sample does not contain VITT antibodies, and (b) detecting the presence or absence of the antibody-uncomplexed PF4 polypeptide complexes, wherein the presence of the antibody-uncomplexed PF4 polypeptide complexes indicates that the mammal has VITT, and wherein the absence of the antibody-uncomplexed PF4 polypeptide complexes indicates that the mammal does not have VITT. The sample can be a blood sample. The mammal can be a human. The detecting can include using an ELISA. The method can further include, when the presence of the antibody-uncomplexed PF4 polypeptide complexes is detected, administering to the mammal IVIg, a non-heparin anticoagulant, a corticosteroid, TPE treatment, or a combination thereof to treat the VITT. The method can further include, when the presence of the antibody-uncomplexed PF4 polypeptide complexes is detected, monitoring the mammal for platelet counts or D-dimer levels to assess the severity or progression of the VITT.

In another aspect, this document features a method for treating VITT. The method can include, or consist essentially of, administering IVIg, a non-heparin anticoagulant, a corticosteroid, TPE treatment, or a combination thereof to a mammal identified as having VITT based at least in part on detection of antibody-uncomplexed PF4 polypeptide complexes in a sample from the mammal that was contacted with an uncomplexed PF4 polypeptide. The sample can be a blood sample. The mammal can be a human. The detection can include using an ELISA. The method can further include monitoring the mammal for platelet counts or D-dimer levels to assess the severity or progression of the VITT.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show that PF4/polyanion ELISAs were sensitive but highly non-specific, while an un-complexed PF4 ELISA was both sensitive and specific for the detection of VITT antibodies. Binding of antibodies from five Ad26.COV2.S-associated VITT samples (solid black), one ChAdOx1 nCOV-19-associated VITT sample (hatched), one mRNA-1273-associated VITT sample (vertical lines), two s-HIT samples (horizontal lines), three delayed-onset HIT samples (gray), eight classical HIT samples (open circles), and five ELISA⁺/PEA⁻ samples (squares) to immobilized PF4-polyanion complexes was evaluated using the LIFECODES PF4 IgG immunoassay (FIG. 1A) or the un-complexed PF4 ELISA (FIG. 1B). The dotted line in FIG. 1A designates the positive cut-off for the PF4-polyanion ELISA per the kit manufacturer (0.4 OD). In FIG. 1B, an arbitrary horizontal line indicates the clear separation of results for VITT vs. non-VITT groups. Groups were compared using one-way ANOVA. Mean and standard error are shown. ns, not significant (p=0.5192); ****P<0.0001. Twenty-five healthy control sera had a mean OD of 0.130 (range 0.075-0.233) in the un-complexed PF4 ELISA (data not shown).

FIGS. 2A-2D show that VITT antibodies are strongly ELISA positive, activate PF4-treated platelets, and are persistent. FIG. 2A is a graph plotting the optical density (OD) results of PF4-polyanion ELISA testing of samples from nine patients upon initial presentation with VITT. Eight patients were tested using the LIFECODES PF4 IgG (IgG-specific) assay, while one was tested using the LIFECODES PF4 Enhanced assay that detects IgG, IgA, and IgM (both are FDA-approved, in vitro diagnostic assays for HIT). Mean and standard error are shown. The dotted line represents the positive cut-off of the assay. FIG. 2B is a graph plotting serotonin release assay (SRA) and PEA results for six VITT patients upon initial presentation. Open circles, SRA; closed circles, PEA. Mean and standard error are shown. The solid and dotted lines represent the positive cut-offs for the SRA and PEA, respectively. FIG. 2C is a graph plotting Kaplan Meier curves for time to negative ELISA OD (<0.4 OD) and PEA (<19%). FIG. 2D is a graph plotting Kaplan Meier curves for time to normalization of D-dimer (cut-off varied by testing laboratory, as shown in TABLE 1) and platelet counts (>150,000/μL).

FIGS. 3A-3D show that use of uncomplexed PF4 targets, but not complexed PF4 targets, permit differentiation of VITT after nine-valent HPV vaccination from HIT and spontaneous HIT. FIG. 3A is a graph plotting the reactivity of acute patient sample to PF4-polyanion complexes, evaluated in a HIT ELISA (LIFECODES PF4 IgG assay, Immucor). The acute patient sample and a positive control (a sample from a HIT patient) recognized polyanion-complexed PF4. The dotted line indicates the assay's positive cut-off of 0.4 optical density. FIG. 3B is a graph plotting reactivity of acute patient serum and a positive control (a sample from an Ad26.COV2.S-associated VITT patient) to un-complexed PF4. Both HIT and spontaneous HIT patients tested negative in the un-complexed PF4 ELISA. The solid line indicates the assay's positive cut-off of 1.0 optical density. The spontaneous HIT patient tested here had been reported to be strongly reactive in PF4-polyanion ELISA, with an optical density of 2.7823. Reactivity of a follow-up patient sample in PF4-polyanion ELISA and un-complexed PF4 ELISA is shown in FIG. 3C and FIG. 3D, respectively. NC, normal healthy control serum; IVIg, intravenous immunoglobulin G; HIT, heparin-induced thrombocytopenia; HPV-VITT, VITT after nine-valent HPV vaccine (acute sample); HPV-VITT-F/U, VITT after nine-valent HPV vaccine (follow-up sample); s-HIT, spontaneous HIT; Ad26.COV2.S, VITT (Janssen Johnson & Johnson COVID-19 vaccine-associated VITT).

FIGS. 4A-4C show that the assays described herein can be used to differentiate between mammals having true VITT and those who present with symptoms mimicking VITT but are falsely positive in a PF4/polyanion ELISA. FIGS. 4A and 4B are graphs plotting reactivity of serum samples from a patient with thrombosis/thrombocytopenia in ELISAs utilizing immobilized PF4/polyanion complexes (FIG. 4A) or un-complexed PF4 (FIG. 4B). FIG. 4C is a graph plotting the results of PEA testing to determine whether the patient had platelet-activating antibodies. No platelet activating antibodies were observed in the patient sample, suggesting that the positive PF4/polyanion ELISA result shown in FIG. 4A was a false positive. NC, normal healthy control blood sample; HIT, blood sample from a patient diagnosed with HIT; VITT, blood sample from a patient with confirmed VITT; HDH, high dose heparin; and PEA, PF4-dependent P-selectin expression assay.

### DETAILED DESCRIPTION

This document provides methods and materials for specifically identifying and/or treating mammals (e.g., humans) having VITT, such that they can be distinguished from mammals having s-HIT, for example. In some cases, the methods and materials provided herein also can be used to distinguish mammals having VITT from mammals having HIT, from mammals having non-pathogenic anti-PF4 antibodies, and from heparin-exposed individuals with non-pathogenic anti-PF4 antibodies. The methods and materials provided herein utilize uncomplexed PF4 to specifically detect (e.g., through an ELISA) pathogenic anti-PF4 antibodies associated with VITT. As described in the Examples, an uncomplexed PF4 ELISA had 100% sensitivity and specificity for detection of VITT associated with several different COVID vaccines, while commonly used ELISAs utilizing PF4-polyanion complexes were highly sensitive but very poorly specific.

Any appropriate sample from a mammal can be assessed as described herein to determine whether the mammal has VITT. Examples of samples that can contain pathogenic anti-PF4 antibodies and used as described herein include, without limitation, blood samples (e.g., whole blood, serum, or plasma samples), urine, saliva, sputum, and broncho-alveolar lavage samples. In some cases, a sample can be a whole blood sample.

In some cases, samples such as blood samples (e.g., whole blood, serum, or plasma samples), urine, saliva, sputum, and broncho-alveolar lavage samples can contain non-pathogenic anti-PF4 antibodies. Non-pathogenic anti-PF4 antibodies are not platelet activating antibodies, but they do yield a positive result in PF4-polyanion ELISAs. Such non-pathogenic antibodies can be found in healthy individuals, as more than 4% of healthy people have non-pathogenic anti-PF4 antibodies (Hursting et al., supra). Such antibodies also can be found in heparin-exposed patients who do not develop HIT (Pouplard et al., Circulation 1999, 99(19):2530-2536).

A sample to be assayed as described herein can be from any appropriate mammal. For example, a sample to be tested using the methods and materials described herein can be from a human or another primate, such as a monkey. In some cases, a sample to be tested can be from a dog, a cat, a horse, a cow, a pig, a sheep, a mouse, or a rat. The mammal can have thrombocytopenia, thrombosis, or both thrombocytopenia and thrombosis. In some cases, the mammal can be identified as not having received a heparin treatment within a designated time frame (e.g., within the last 30 days, within the last 30 to 60 days, within the last 60 to 90 days, within the last 90 to 120 days, or within the last 120 to 180 days), such that the mammal can be classified as not having HIT. In some cases, the mammal can be identified as having received heparin treatment within a designated time frame (e.g., within the last 30 days, within the last 30 to 60 days, within the last 60 to 90 days, within the last 90 to 120 days, or within the last 120 to 180 days), such that the mammal can be classified as possibly having HIT. In some cases, the mammal can be identified as possibly harboring PF4/polyanion ELISA positive but non-activating anti-PF4 antibodies. The mammal also can be identified as having received a vaccine (e.g., a COVID-19 vaccine or an HPV vaccine). In some cases, the mammal can be identified as having received a vaccine (e.g., a COVID-19 vaccine or an HPV vaccine) within a certain time frame (e.g., within the last 30 days, within the last 30 to 60 days, within the last 60 to 90 days, within the last 90 to 120 days, or within the last 120 to 180 days).

The methods provided herein can include contacting a sample (e.g., a blood, serum, or plasma sample from a human having thrombocytopenia and/or thrombosis but, at least in some cases, not having HIT) with an uncomplexed PF4 polypeptide, and determining whether antibody-uncomplexed PF4 polypeptide complexes are present in the mixture. If the sample contains VITT antibodies, antibody-uncomplexed PF4 polypeptide complexes can form and be detected. In such cases, the mammal (e.g., human) can be identified as having VITT. In contrast, if the sample does not contain VITT antibodies, antibody-uncomplexed PF4 polypeptide complexes will not form and therefore will not be detected. In such cases, the mammal (e.g., human) can be identified as not having VITT.

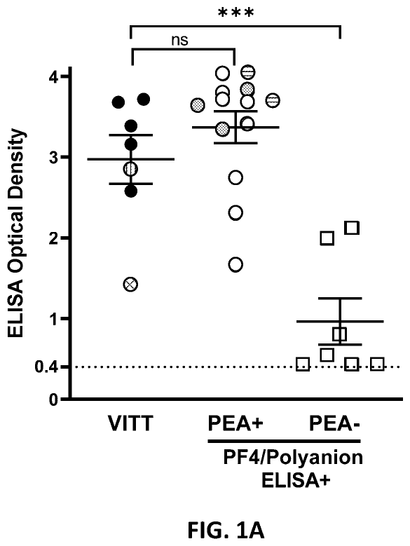

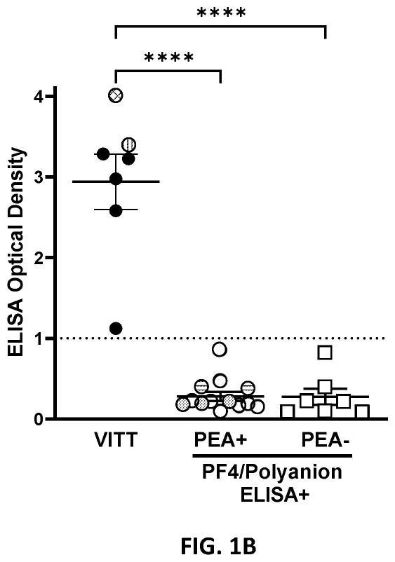

In some cases, the methods provided herein can distinguish a mammal with VITT antibodies from a mammal with non-pathogenic anti-PF4 antibodies. For example, if a mammal having non-pathogenic anti-PF4 antibodies receives a vaccine and subsequently develops thrombosis and/or thrombocytopenia, the thrombosis and/or thrombocytopenia may or may not be related to the vaccine (that is, the thrombosis and/or thrombocytopenia may be due to VITT, or may just be coincidental with administration of the vaccine). A PF4-polyanion ELISA would be positive (see, FIG. 1A), but would not differentiate between pathogenic VITT antibodies and the non-pathogenic anti-PF4 antibodies of the mammal. In contrast, the uncomplexed PF4 ELISA provided herein can be used to distinguish between VITT antibodies and non-pathogenic anti-PF4 antibodies (see., e.g., FIG. 1B).

The uncomplexed PF4 polypeptide used in the methods provided herein can be a full length PF4 polypeptide or a fragment of a PF4 polypeptide. Representative examples of amino acid sequences for PF4 polypeptides are set forth in NCBI Reference Sequences NP_002610 (e.g., version NP_002610.1; SEQ ID NO:1), and NP_001350281 (e.g., version NP_001350281.1; SEQ ID NO:2). These sequences are encoded by transcript variants, with variant 2 using an alternate splice site that results in the use of an alternate start codon, compared to variant 1. Variant 2 encodes a longer protein (isoform 2; SEQ ID NO: 2) with a distinct N-terminus, compared to isoform 1. Further, SEQ ID NO: 1 is a PF4 precursor sequence; the mature isoform 1 PF4 amino acid sequence is set forth in amino acids 32-101 (underlined in SEQ ID NO:1).

(SEQ ID NO: 1)
MSSAAGFCASRPGLLFLGLLLLPLVVAFASAEAEEDGDLQCLCVK
TTSQVRPRHITSLEVIKAGPHCPTAQLIATLKNGRKICLDLQAPL
YKKIIKKLLES
(SEQ ID NO: 2)
MITATLNGEPAECLATVPGAAPAPPTWLEQLLSGGGVIYAEAEED
GDLQCLCVKTTSQVRPRHITSLEVIKAGPHCPTAQLIATLKNGRK
ICLDLQAPLYKKIIKKLLES

In some cases, a PF4 polypeptide can be coupled to a tag. For example, a PF4 polypeptide can be tagged with, for example, biotin, polyhistidine, glutathione-S-transferase, calmodulin-binding protein, or the like (e.g., for immobilization and subsequent use in the methods provided herein).

When an uncomplexed PF4 polypeptide fragment is used in the methods provided herein, the fragment can have any appropriate length. For example, an uncomplexed PF4 fragment can have a length of about 10 to about 100 amino acids (e.g., about 10 to about 20, about 20 to about 40, about 30 to about 50, about 40 to about 60, about 50 to about 70, about 60 to about 80, about 70 to about 90, or about 80 to about 100 amino acids).

Any appropriate method can be used to detect the presence or absence of antibodies to uncomplexed PF4 polypeptide (also referred to herein as detecting the presence or absence of antibody-uncomplexed PF4 polypeptide complexes). In some cases, for example, an ELISA method can be used to determine whether antibody-uncomplexed PF4 polypeptide complexes are present in an assay sample. In some cases, the presence or absence of antibody-uncomplexed PF4 polypeptide complexes can be detected using immunoturbidometry, chemiluminescence, a lateral flow assay (LFA), surface plasmon resonance, or a microfluidics based assay.

In some cases, the methods provided herein can further include steps for monitoring and/or treating mammals (e.g., humans) identified as having VITT or as not having VITT. In some cases, for example, rapid initiation of treatment with intravenous immunoglobulin G (IVIg), non-heparin anticoagulants, corticosteroids, and/or therapeutic plasma exchange (TPE) can be used to treat mammals (e.g., humans) identified as having VITT (see, e.g., hematology.org/covid-19/vaccine-induced-immune-thrombotic-thrombocytopenia). In contrast, a mammal (e.g., a human) identified as having HIT or s-HIT as described herein can be treated in the acute phase by administering non-heparin anticoagulants until platelet recovery, after which a vitamin K antagonist/direct oral anticoagulant can be used (see, e.g., Cuker et al., Blood Adv. 2018, 2(22):3360-3392). A mammal (e.g., a human) identified as being a heparin-exposed mammal or a healthy mammal that is PF4/polyanion ELISA positive but with non-activating antibodies can be instructed to proceed without any specific treatment. Individuals identified as having VITT as described herein also can be sent down a different path related to receipt of follow-on COVID-19 vaccine doses/boosters. In some cases, patients identified as having VITT as described herein may not be administered the same type of vaccine as they previously received, but rather may be administered vaccines from other classes. For example, ChAdOx01 nCOV-19-associated VITT patients can be instructed to receive mRNA COVID vaccines (Schönborn et al., New Engl J Med. 2021, 385(19):1815-1816) for subsequent vaccinations (e.g., boosters). In contrast, individuals identified as not having VITT (e.g., individuals identified as having another category of thrombosis/thrombocytopenia, such as HIT or s-HIT, healthy individuals with non-pathogenic anti-PF4 antibodies, individuals with thrombosis/thrombocytopenia due to unknown causes, or heparin-exposed individuals with non-pathogenic anti-PF4 antibodies) using the methods described herein would not be expected to be adversely affected, safety-wise, by a second or booster dose of the same class of COVID-19 vaccine that they previously received. Further, due to the persistence of anti-PF4 antibodies in the majority of VITT patients even 6 months after acute presentation, as well as the recurrence and persistence of thrombocytopenia in some VITT patients, individuals identified as having VITT as described herein can be treated for a longer period of time with an anticoagulant, may be subjected to more frequent monitoring of platelet counts, and in some cases, may be monitored by laboratory assessment of factors such as D-dimer levels.

The invention will be further described in the following examples, which do not limit the scope of the invention described in the claims.

### EXAMPLES

### Example 1—Comparing Uncomplexed PF4 ELISA to Immucor ELISA

To carry out uncomplexed PF4 ELISA, ELISA plates (Thermo Scientific) were incubated with recombinant PF4 (Protein Foundry; 10 μg/mL), and plates were washed three times with phosphate-buffered saline (PBS) pH 7.4/0.1% TWEEN®-20 and blocked with SUPERBLOCK™ T20 (Thermo Scientific). Serum or plasma samples were incubated on the plates at 1:50 dilution for 60 minutes, followed by four washes with PBS/0.1% TWEEN®-20. After a 45-minute incubation with 50 μL of alkaline phosphatase-conjugated goat anti-human IgG Fc antibody (Jackson Immunoresearch) at a dilution of 1:5000, four additional washes were performed using PBS/0.1% TWEEN®-20. Colorimetric detection was performed using p-nitrophenyl phosphate (pNPP) substrate, and the optical density (OD; 405 nm minus 492 nm) at 30 minutes was recorded. For the Immucor ELISA, the LIFECODES PF4 IgG ELISA (an FDA-approved HIT ELISA kit) was used per manufacturer instructions.

These studies demonstrated that in the uncomplexed PF4 ELISA, samples from VITT patients had significantly higher OD than samples from patients with HIT (including its severe subtype), s-HIT, or frequently encountered non-pathogenic antibodies that are positive in PF4-polyanion ELISAs (FIG. 1A). In contrast, in the Immucor ELISA, although VITT samples were associated with high OD, they were highly overlapping with the ODs obtained with HIT and s-HIT samples (FIG. 1B).

### Example 2—Persistence and Specific Detection of AD26.COV2.S-Related VITT Antibodies

### Methods and Materials

Patient samples and clinical course: Blood samples were obtained from nine patients with VITT after Ad26.COV2.S vaccination, and also from patients with ChAdOx1 nCOV-19-associated VITT (n=1), mRNA-1273 associated VITT (n=1) classical HIT (n=8), delayed-onset HIT (3), s-HIT (n=2), and non-activating PF4/polyanion ELISA-positive antibodies (n=7). Clinical and laboratory information was extracted from patient medical records.

Laboratory studies: LIFECODES PF4 IgG (Immucor; Norcross, GA), an IgG-specific PF4-polyanion ELISA assay, was used per the manufacturer's instructions. An ELISA developed to evaluate antibody binding to uncomplexed PF4 was performed as described in Example 1. The PF4-dependent PEA was performed as described elsewhere (Samuelson Bannow et al., Blood 2021, 137:1082-1089). Serotonin release assay (SRA), platelet count, and D-dimer testing also were performed. Normal ranges for the LIFECODES PF4 IgG assay, SRA, and PEA were <0.4 OD, <20% serotonin release, and <19% P-selectin expression, respectively.

Statistics: One-way ANOVA was used to compare the three groups of VITT, HIT, and ELISA+/PEA− patient samples. One patient who had a negative PEA result followed by a positive result on longer follow-up had the earlier negative result excluded from the analysis. This patient also had two negative PF4-polyanion ELISA results (one from a post-therapeutic plasma exchange sample) followed by numerous samples with positive results. These two negative results were excluded for the purpose of Kaplan-Meier curve calculation. Time to normalization of platelet count and D-dimer was calculated as the time to a normal result that was not followed by an abnormal result at a longer follow-up time point. Kaplan-Meier curves were calculated using Turnbull's method that accounts for both right and left censoring.

### Results

Presentation, clinical course, and management: The Ad26.COV2.S-associated VITT patient cohort included five males and four females with a median age of 46 years (range 28-51; TABLE 1). Patients were followed for a median of 190 days (range 166-202). All patients presented with thrombocytopenia and thrombosis, and the median time from Ad26.COV2.S vaccination to onset of symptoms was 11 days (range 6-14 days). At presentation, the median platelet count was 54,000/μL (range, 9000-126,000/μL), and D-dimer was extremely high in all patients (TABLE 1). Thrombosis at uncommon locations predominated, with cerebral venous sinus thrombosis (CVST) and splanchnic vein thrombosis seen in five and four patients, respectively (some patients had thrombosis at both sites; TABLE 1). Five patients developed pulmonary embolism (PE). Only two of the nine patients presented with non-CVST/splanchnic vein thromboses, and both developed pulmonary embolism and deep venous thrombosis (DVT). All patients were treated with high-dose intravenous immunoglobulin G (IVIg) and a direct thrombin inhibitor during the acute episode, while eight of the nine patients also received steroids (TABLE 1). After recovery, patients received long-term anticoagulation with direct oral anticoagulants (DOACs). Three patients experienced a recurrence of thrombocytopenia.

Two of the three received additional IVIg therapy; no platelet increment was seen in one of these two patients, and the other patient required IVIg re-dosing for platelet normalization. In the third patient, thrombocytopenia resolved without additional intervention. Three additional patients had long term persistence of thrombocytopenia, one of whom was treated with IVIg, rituximab, and therapeutic plasma exchange. At last follow-up, all but two patients were on DOACs (stopped at 6 and 7.5 months). No patient had recurrence of thrombosis and there were no fatalities.

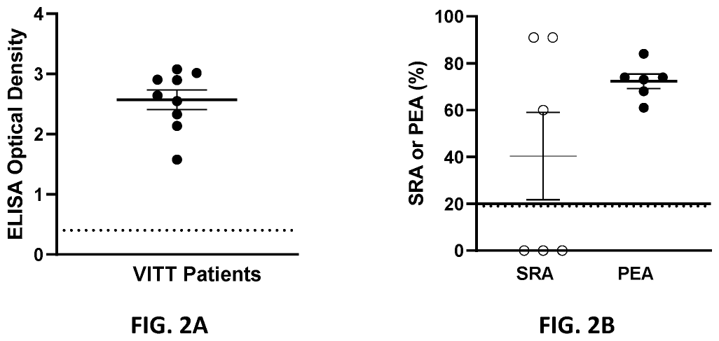

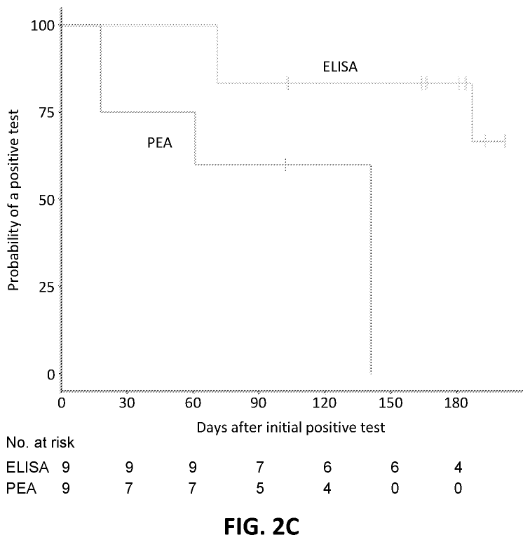

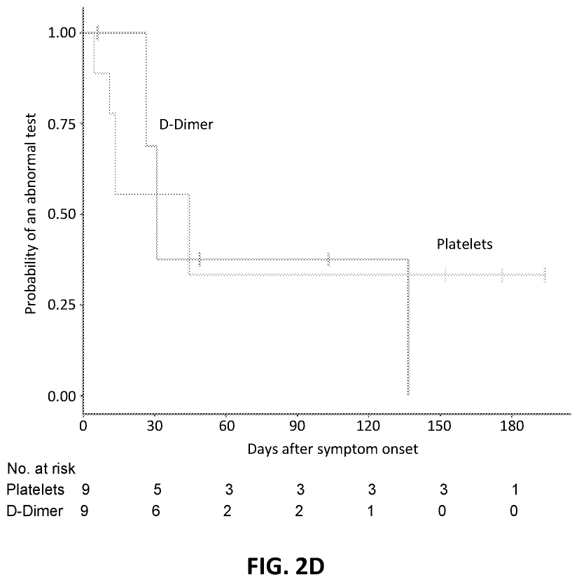

Presence and prevalence of anti-PF4 antibodies: All patients had strongly positive PF4-polyanion ELISA results (OD>1.0), and eight patients demonstrated ODs>2.0 (FIG. 2A). Two patients tested negative with an automated, latex enhanced immuno-turbidimetric assay (HemosIL HIT-Ab [PF4-H]; Instrumentation Laboratory, Bedford, MA). Eight of the nine VITT patients were tested by SRA using heparin-treated platelets, with only four testing positive. Available blood samples from six patients were tested in the PEA, a functional HIT assay that uses PF4 rather than heparin-treated platelets (Samuelson Bannow et al., supra). All six samples activated PF4-treated platelets in this assay, while only three were positive by the SRA (FIG. 2B). Longitudinal samples from the nine VITT patients were tested in PF4-polyanion ELISA and PEA. At last follow-up, seven of the nine patients had positive ELISA results, all with OD>1.0. One of the two patients who became seronegative had received four doses of rituximab (at 375 mg/m²) and therapeutic plasma exchange (TPE) treatment. The probability of a positive ELISA result was still high after five months of the acute event (FIG. 2C). In contrast, eight of the nine patients were PEA-negative at their last follow-up, with only one positive result (46%) 103 days after acute presentation. In contrast to ELISA, the probability of a positive PEA was very low five months after the acute event (FIG. 2C).

Persistence of thrombocytopenia and D-dimer elevation: Platelets were mildly to moderately decreased in three patients even after five months of follow-up (176, 194, and 152 days), and were at 141,000/μL, 149,000/μL, and 89,000/μL, respectively, while D-dimer levels had normalized in that time frame (FIG. 2D). One of the three patients with persistent thrombocytopenia had pre-existing thrombocytopenia secondary to hepatic cirrhosis. Thus, it was deemed unlikely that persistence was due to VITT antibodies in this patient.

Specific detection of VITT antibodies: To compare the ability of current HIT ELISAs that employ PF4-polyanion targets and a novel uncomplexed PF4 ELISA to distinguish VITT from s-HIT, classical HIT (including the severe delayed-onset type), and non-pathogenic anti-PF4 antibodies, acute samples from five Ad26.COV2.S-associated VITT, one ChAdOx1 nCOV-19-associated VITT, one mRNA-1273-associated VITT, two s-HIT, eight classical HIT, 3 delayed-onset HIT patients and seven ELISA+/PEA-patient samples were tested. Clinical and laboratory characteristics of the HIT, delayed-onset HIT and s-HIT cohorts are presented in TABLE 2. s-HIT patients were heterogeneous in presentation, with one patient developing disease after knee arthroplasty and the other after several days of presumed infection characterized by nausea and fatigue. As shown in FIG. 1A, the PF4-polyanion assay was highly non-specific for the detection of VITT antibodies, with samples from VITT, HIT, delayed-onset HIT and s-HIT patients producing strong reactions. Although non-pathogenic anti-PF4 antibodies produced lower ODs, they were still positive in the PF4/polyanion assay. In contrast, when this sample cohort was tested in an experimental ELISA assay using uncomplexed rather than polyanion-complexed PF4 targets (FIG. 1B), clear differentiation of VITT from s-HIT, classical HIT, delayed-onset HIT, and ELISA+/PEA-samples was seen, with anti-PF4 antibodies from VITT patients producing significantly higher optical densities relative to the non-VITT group.

TABLE 1
Demographic, laboratory, and clinical features of Ad26.COV2.S-associated VITT patients

| | | | Symptom | | | | | | |
|---|---|---|---|---|---|---|---|---|---|
| | | | onset after | | D-dimer | | Treatment | | |
| Patient | Age | | vaccination | Platelets^{#} | (ng/mL | Thrombotic | during acute | Subsequent | |
| No. | (yrs) | Sex | (days) | (per μL) | FEU) | features | episode | treatment | Outcome |
| 1 | 46 | M | 13 | 54,000 | 28,980 | PE and DVT | Bivalirudin, IVIg, | Apixaban | Alive |
| | | | | | | | and prednisone | | |
| 2 | 40 | F | 6 | 20,000 | 27,150 | PE and CVST | Bivalirudin, IVIg, | Rivaroxaban | Alive |
| | | | | | | | and prednisone | | |
| 3 | 34 | F | 14 | 126,000 | 39,930 | CVST | Bivalirudin, IVIg, | Apixaban | Alive |
| | | | | | | | and prednisone | | |
| 4 | 48 | F | 14 | 13,000 | 112,073 | CVST and | Argatroban, IVIg, | IVIg and | Alive |
| | | | | | | splanchnic vein | and prednisone | Apixaban | |
| | | | | | | thrombosis | | | |
| 5 | 28 | M | 10 | 66,000 | 22,546 | PE, CVST and | Argatroban and | Apixaban | Alive |
| | | | | | | splanchnic vein | IVIg | | |
| | | | | | | thrombosis | | | |
| 6 | 51 | F | 7 | 55,000 | >20,000 | PE and CVST | Argatroban, IVIg, | Apixaban | Alive |
| | | | | | | | and prednisone | | |
| 7 | 48 | M | 11 | 99,000 | 15,100* | PE and DVT | Argatroban, IVIg, | IVIg and | Alive |
| | | | | | | | and prednisone | Apixaban | |
| 8 | 33 | M | 8 | 31,000 | >10,000** | Splanchnic vein | Argatroban, IVIg, | Apixaban | Alive |
| | | | | | | thrombosis | and dexamethasone | | |
| 9 | 50 | M | 13 | 9,000 | 5,270* | Splanchnic vein | Argatroban, IVIg, | IVIg, TPE, | Alive |
| | | | | | | thrombosis | and prednisone | rituximab, | |
| | | | | | | | | warfarin | |

^{#}Lower limit of the platelet reference range was 150,000/μL for all laboratories.
D-dimer reference range was <500 ng/mL FEU except for *(<400 ng/mL FEU) and **(<230 ng/mL DDU).
IVIg, intravenous immunoglobulin G; TPE, therapeutic plasma exchange; FEU, fibrinogen equivalent units; DDU, D-dimer units.
Clinical information on the ChAdOx1 nCoV-19 associated and mRNA-1273-associated VITT patient are described elsewhere (Bayas et. al., Lancet 2021, 397(10285): e11; and Sangli et al., Ann Intern Med. 2021, 174(10): 1480-1482, respectively).

#Lower limit of the platelet reference range was 150,000/μL for all laboratories. D-dimer reference range was <500 ng/mL FEU except for *(<400 ng/mL FEU) and **(<230 ng/ml DDU). IVIg, intravenous immunoglobulin G; TPE, therapeutic plasma exchange; FEU, fibrinogen equivalent units; DDU, D-dimer units. Clinical information on the ChAdOx1 nCOV-19-associated and mRNA-1273-associated VITT patient are described elsewhere (Bayas et al., Lancet 2021, 397(10285):e11; and Sangli et al., Ann Intern Med. 2021, 174(10):1480-1482, respectively).

TABLE 2
Platelet nadir and thrombosis characteristics
of HIT, delayed-onset HIT and s-HIT cohorts

| | | | | |
|---|---|---|---|---|
| | | | Platelet Nadir | |
| N | Age | Sex | (/uL) | Thrombosis* |

″Classical″ HIT

| | | | | |
|---|---|---|---|---|
| 1 | 87 | M | 87,000 | Y |
| 2 | 57 | M | 39,000 | N |
| 3 | 76 | M | 134,000 | Y |
| 4 | 50 | F | 34,000 | Y |
| 5 | 48 | M | 7,000 | Y |
| 6 | 58 | M | 53,000 | N |
| 7 | 73 | M | 95,000 | N |
| 8 | 60 | F | 95,000 | Y |

Delayed-Onset HIT

| | | | | |
|---|---|---|---|---|
| 9 | 66 | M | 40,000 | Y |
| 10 | 75 | M | 37,000 | Y |
| 11 | 47 | M | 8,000 | Y |

s-HIT

| | | | | |
|---|---|---|---|---|
| 12 | 30 | M | 41,000 | Y |
| 13 | 70 | F | 19,000 | Y |

*Y, thrombosis noted;
N, no thrombosis noted.

### Example 3—Human Papilloma Virus Vaccine and VITT Antibody Induction

A 25-year-old female without a history of heparin exposure presented with thrombocytopenia, thrombosis, and elevated D-dimer levels ten days after receiving a nine-valent human papillomavirus (HPV) vaccination (GARDASIL®, Merck and Co.; Johansen et al., supra). The patient's samples activated platelet factor 4 (PF4)-treated platelets and recognized PF4-polyanion complexes in HIT ELISA (PF4-polyanion enzyme-linked immunosorbent assay). Thrombosis sites included the right internal iliac vein and bilateral pulmonary emboli. When it was recognized that the patient had a VITT-like presentation, she was treated with intravenous immunoglobulin G (IVIg), to which she responded promptly. While these clinical and laboratory features were highly consistent with VITT, they overlapped with spontaneous HIT, an IVIg-responsive condition where thrombosis and thrombocytopenia develop in the absence of proximate heparin exposure (Warkentin et al., Blood 123(23):3651-3654, 2014; Irani et al., Transfusion 59(3):931-934, 2019; Mohanty et al., J Thromb Haemost. 17(5):841-844, 2019; and Hwang et al., Platelets 1-5.2-5, 2020).

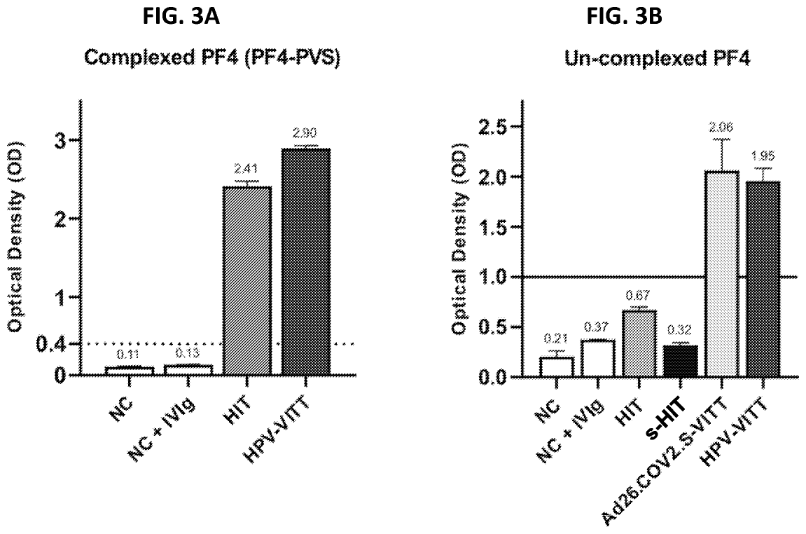

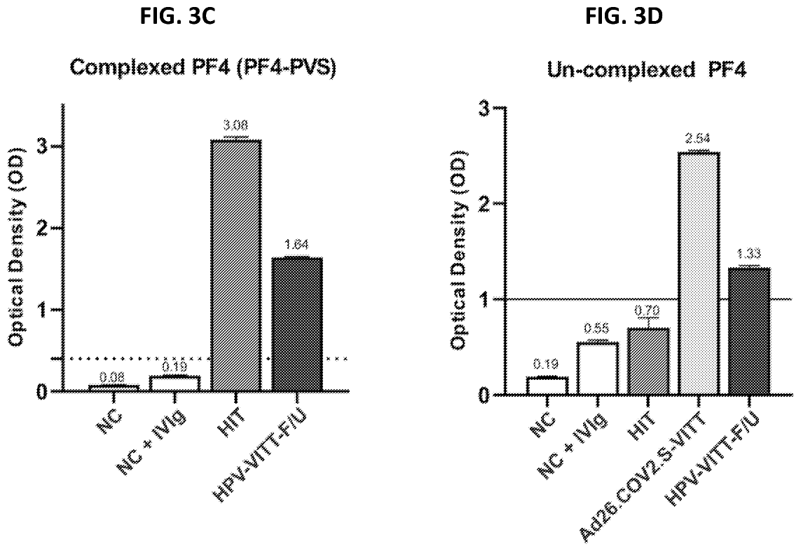

A limitation in the preliminary causality assessment linking nine-valent HPV vaccination and the thrombotic thrombocytopenia reaction was the lack of tests specific for VITT at the time of presentation, since PF4-polyanion ELISAs and PF4-platelet-based functional assays detect VITT (Kanack et al., Am J Hematol. 97(5):519-526, 2022), HIT (Samuelson Bannow et al., Blood 137(8):1082-1089, 2021), and spontaneous HIT antibodies (Irani et al., supra). As described herein, however, an un-complexed PF4 ELISA can sensitively and specifically detect VITT antibodies and differentiate them from antibodies that cause spontaneous HIT and HIT. Thus, to further study the antibodies in this patient, serum was evaluated in the un-complexed PF4 ELISA. In addition, the patient's sample was evaluated in a HIT (PF4-polyanion) ELISA for comparison (LIFECODES PF4 IgG, Immucor). Since the sample was obtained after IVIg therapy, levels of IgG were quantified (17.88 mg/mL; IgG Human ELISA Kit, Thermo Fisher) and a normal control serum (NC) was spiked with IVIg (Privigen, CSL Behring) to attain equivalent IgG levels for use as a control. Like sera from HIT patients, antibodies from the patient strongly recognized PF4-polyanion complexes (FIG. 3A). In contrast to both HIT and spontaneous HIT sera, antibodies from this patient also strongly recognized un-complexed PF4 in this VITT-specific assay (FIG. 3B). A sample obtained 12 months after acute presentation continued to test strongly positive (>1.0 OD) in both PF4-polyanion (FIG. 3C) and un-complexed PF4 ELISAs (FIG. 3D), although it was not platelet activating (4% in a PEA), consistent with reports that demonstrated persistent positivity of VITT antibodies by ELISA but loss of platelet activating ability with long-term follow up (Kanack et al., supra; Samuelson Bannow et al., supra; and Schonborn et al., N Engl J Med. 385(19):1815-1816, 2021). The patient was clinically stable, with no thrombosis/thrombocytopenia and a normal platelet count of 246,000/μL at the time of follow-up sampling. A negative PF4-polyanion ELISA result obtained four months prior to HPV vaccination minimized the possibility that pre-existing anti-PF4 antibodies mediated the adverse reaction (Johansen et al., supra). Thus, these studies demonstrated that blood samples from the HPV vaccinated patient who developed thrombosis and thrombocytopenia reacted with un-complexed PF4 in the ELISA, confirming VITT.

### Example 4—Differentiating Between True VITT and False Positive VITT Presentations

Up to 5% of the general population may have positive results in PF4-polyanion ELISAs. As noted above, non-pathogenic antibodies can be found in healthy individuals, as more than 4% of non-heparin treated people have non-pathogenic anti-PF4 antibodies (Hursting et al., supra). One such individual was identified as described below, highlighting the fact that the methods described herein can differentiate between mammals that have true VITT and those that may present with symptoms that mimic VITT but are falsely positive in PF4/polyanion ELISA.

A 27-year-old male with no history of thrombosis presented at the hospital with anterior midsternal chest tightness and shortness of breath that had been persistent since the previous day. A routine physical examination indicated tachycardia. The patient reported no known previous exposure to heparin, but indicated that they had received a second dose of the mRNA-1273 vaccine two weeks prior to their presentation. Laboratory testing on the day of the patient's hospital admission revealed elevated levels of D-dimer (5,658 ng/mL; reference range: 0-500 ng/mL) and Troponin I (2.294 ng/ml; reference range: 0.000-0.033 ng/mL). Platelet counts were within the normal reference range (150,000-400,000/μL) upon admission, but reached a nadir of 96,000/μL the following day. Imaging by computerized tomography angiography of the chest revealed a large, acute saddle pulmonary embolus extending into the left and right pulmonary arteries, resulting in right heart strain. A duplex Doppler ultrasound of the lower extremities showed a left lower extremity (LLE) deep venous thrombosis (DVT).

Subsequently, the patient was administered heparin (18 U/kg/hr) and underwent a successful bilateral thrombectomy. Two days following his hospital admission, the patient was transitioned from heparin to apixaban and later discharged from the hospital, and has had no recurrence of thrombosis or thrombocytopenia.

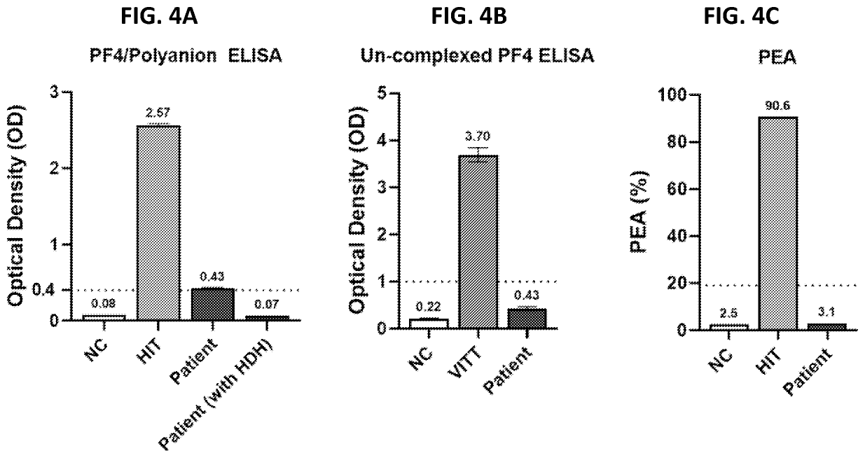

Given the proximity between the patient receiving the mRNA-1273 booster vaccine, and occurrence of thrombocytopenia and thromboses, VITT was retrospectively suspected as a potential cause. Follow-up serum samples were obtained from the patient about four weeks post-hospital admission, and were tested in a HIT ELISA utilizing immobilized PF4/polyanion complexes as a target (LIFECODES PF4 IgG; Immucor). The patient was positive in this test, with an optical density (OD) of 0.43 (the negative cutoff is <0.40), which was inhibited in the presence of high concentrations of heparin as would be expected for VITT antibodies (FIG. 4A). Since ELISAs that utilize PF4 complexed to polyanions cannot differentiate between antibodies associated with VITT and those in individuals who may have non-pathogenic/non-platelet activating antibodies, the patient serum also was evaluated using the un-complexed PF4 ELISA described herein (see, also, Kanack et al., supra). In contrast to an individual who developed VITT after receiving the ChAdox1 CoV-19 vaccine and was strongly reactive to un-complexed PF4 (OD 3.7; negative cutoff <1.0), the patient described here tested negative (OD 0.43) in the un-complexed ELISA (FIG. 4B). However, since the patient tested weakly positive by PF4/polyanion ELISA, confirmatory functional (platelet-activation based) PEA testing was performed to determine if the patient had platelet-activating antibodies. As shown in FIG. 4C, no platelet activating antibodies were observed in the patient sample, confirming that the PF4/polyanion ELISA result was falsely positive for VITT.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for distinguishing vaccine-induced immune thrombotic thrombocytopenia (VITT) from spontaneous heparin-induced thrombocytopenia (s-HIT), wherein said method comprises:
(a) contacting an uncomplexed PF4 polypeptide with a sample from a mammal that (i) has thrombocytopenia, thrombosis, or both thrombocytopenia and thrombosis, and (ii) does not have heparin-induced thrombocytopenia (HIT), wherein antibody-uncomplexed PF4 polypeptide complexes form if said sample contains VITT antibodies, and wherein antibody-uncomplexed PF4 polypeptide complexes do not form if said sample does not contain VITT antibodies, and
(b) detecting the presence or absence of antibody-uncomplexed PF4 polypeptide complexes, wherein the presence of said antibody-uncomplexed PF4 polypeptide complexes indicates that said mammal has VITT, and wherein the absence of said antibody-uncomplexed PF4 polypeptide complexes indicates that said mammal has s-HIT.

2. The method of claim 1, wherein said sample is a blood sample.

3. The method of claim 1, wherein said mammal is a human.

4. The method of claim 1, wherein said detecting comprises using an enzyme-linked immunosorbent assay (ELISA).

5. The method of claim 1, further comprising, when the presence of said antibody-uncomplexed PF4 polypeptide complexes is detected, administering to said mammal intravenous immunoglobulin G (IVIg), a non-heparin anticoagulant, a corticosteroid, therapeutic plasma exchange (TPE) treatment, or a combination thereof to treat said VITT.

6. The method of claim 1, further comprising, when the presence of said antibody-uncomplexed PF4 polypeptide complexes is detected, monitoring said mammal for platelet counts or D-dimer levels to assess the severity or progression of said VITT.

7. A method comprising:
(a) contacting an uncomplexed PF4 polypeptide with a sample from a mammal that (i) has thrombocytopenia, thrombosis, or both thrombocytopenia and thrombosis, and (ii) does not have HIT, wherein antibody-uncomplexed PF4 polypeptide complexes form if said sample contains VITT antibodies, and wherein antibody-uncomplexed PF4 polypeptide complexes do not form if said sample does not contain VITT antibodies, and
(b) detecting the presence or absence of antibody-uncomplexed PF4 polypeptide complexes, wherein the presence of said antibody-uncomplexed PF4 polypeptide complexes indicates that said mammal has VITT, and wherein the absence of said antibody-uncomplexed PF4 polypeptide complexes indicates that said mammal does not have VITT.

8. The method of claim 7, wherein said sample is a blood sample.

9. The method of claim 7, wherein said mammal is a human.

10. The method of claim 7, wherein said detecting comprises using an ELISA.

11. The method of claim 7, further comprising, when the presence of said antibody-uncomplexed PF4 polypeptide complexes is detected, administering to said mammal IVIg, a non-heparin anticoagulant, a corticosteroid, TPE treatment, or a combination thereof to treat said VITT.

12. The method of claim 7, further comprising, when the presence of said antibody-uncomplexed PF4 polypeptide complexes is detected, monitoring said mammal for platelet counts or D-dimer levels to assess the severity or progression of said VITT.

13. A method for treating VITT, wherein said method comprises administering IVIg, a non-heparin anticoagulant, a corticosteroid, TPE treatment, or a combination thereof to a mammal identified as having said VITT based at least in part on detection of antibody-uncomplexed PF4 polypeptide complexes in a sample from said mammal that was contacted with an uncomplexed PF4 polypeptide.

14. The method of claim 13, wherein said sample is a blood sample.

15. The method of claim 13, wherein said mammal is a human.

16. The method of claim 13, wherein said detection comprised using an ELISA.

17. The method of claim 13, further comprising monitoring said mammal for platelet counts or D-dimer levels to assess the severity or progression of said VITT.
